(19) United States
(12) Patent Application Publication
(10) Pub. No.: US 2024/0324960 A1
(43) Pub. Date: Oct. 3, 2024

(54) RF BIOSENSING SYSTEM USING AN RF SENSOR WITH MICRONEEDLE

(71) Applicant: KIMS BIO LAB CO., LTD., Gwangju-si (KR)

(72) Inventors: Nam Young KIM, Gwangju-si (KR); EunSeong KIM, Seoul (KR); Wei YUE, Seoul (KR)

(73) Assignee: KIMS BIO LAB CO., LTD., Gwangju-si (KR)

(21) Appl. No.: 18/617,785

(22) Filed: Mar. 27, 2024

(30) Foreign Application Priority Data
Mar. 28, 2023 (KR) 10-2023-0040825
Jul. 4, 2023 (KR) 10-2023-0086810
Dec. 27, 2023 (KR) 10-2023-0193710

(51) Int. Cl. A61B 5/00; A61B 5/145; A61B 5/1486; A61L 31/04; A61L 31/14

(52) U.S. Cl. CPC A61B 5/685; A61B 5/14532; A61B 5/14865; A61L 31/045; A61L 31/145

(57) ABSTRACT

An RF biosensing system using an RF biosensor with glucose-responsive hydrogel-based microneedles can minimally invasively contact the epidermis/dermis/hypodermis of the skin or the skin surface/fat/muscle in vivo by using glucose-responsive hydrogel-based microneedles provided on the bottom of a flexible substrate of an RF biosensor using resonant frequencies within 400˜3,000 MHZ, detect and transmit the sensing signal to a reader antenna and a reader antenna sensing circuit through a readout coil by RF biosensing and wireless transmission through EM coupling of RF biosensors, and measure the sensing signals by a vector network analyzer (VNA), thereby providing biosensing of proteins, glucose, and fats within the skin in accordance with changes in capacitances of the sensing antenna circuit (LC resonator) of the RF biosensor, resonant frequency shift of the LC resonator, and changes in S-parameters.

### CROSS-REFERENCE TO RELATED APPLICATION(S)

This application claims priority to Korean Patent Application No. 10-2023-0193710, filed on Dec. 27, 2023, Korean Patent Application No. 10-2023-0086810, filed on Jul. 4, 2023, and Korean Patent Application No. 10-2023-0040825, filed on Mar. 28, 2023, with the Korean Intellectual Property Office, the disclosures of which are herein incorporated by reference in their entireties.

### BACKGROUND OF THE INVENTION

### Field of the Invention

The present invention relates to an RF biosensing system having glucose-responsive hydrogel-based microneedles, which minimally invasively contact the epidermis/dermis/hypodermis of the skin in vivo or the skin surface (on-site)/fat/muscle in vivo, detect and transmit the sensing signal to a reader antenna and a reader antenna sensing circuit through a readout coil by RF biosensing and wireless transmission through EM coupling of RF sensors using the difference in resonant frequencies within 400˜3,000 MHz, and measure the sensing signals by a vector network analyzer (VNA), thereby providing biosensing of proteins, glucose, and fats within the skin in accordance with changes in capacitances of the sensing antenna circuit (LC resonator) of the RF biosensor, resonant frequency shift of the LC resonator, and changes in S-parameters.

### Background Art

Carbohydrates consumed by a human are metabolized into glucose and transported into the blood. Insulin secreted by the pancreas supplies cells and acts on an energy source. Diabetes mellitus can occur due to genetic factors, obesity, irregular eating habits (excessive sugar and fat intake, alcohol consumption), lack of exercise, stress, and pre-existing diseases (such as hypothyroidism, pneumonia, and pancreatitis). Diabetes mellitus is characterized by hyperglycemia, where blood sugar exceeds the normal range and the concentration of blood glucose is increased due to insufficient insulin secretion from the pancreas or malfunctioning of insulin. Diabetes mellitus is a complex disease affecting various tissues of the body due to complications like hypertension (cerebral stroke), diabetic retinopathy, myocardial infarction (angina), chronic renal failure, neuropathy, and so on. According to the World Health Organization (WHO), there were approximately 347 million diabetes patients in 2013, and the number is increasing.

Diabetes mellitus is diagnosed by measuring blood glucose levels and glycated hemoglobin levels. Blood glucose test is performed using a method of extracting venous blood, separating only clear serum components of the upper layer after settling the clotted blood, and measuring the glucose concentration, rather than using whole blood glucose test from a fingertip prick.

### *Standard Blood Glucose Criteria

1) Plasma glucose less than 100 mg/dL after at least 8 hours of fasting.
2) Plasma glucose less than 140 mg/dL 2 hours after a 75 g oral glucose tolerance test.

### *Criteria for Diabetes Diagnosis

1) Symptoms of diabetes (polyuria, polydipsia, and weight loss) and a blood glucose level of 200 mg/dL or higher, regardless of meal times.
2) Glycated hemoglobin (HbA1c) level of 6.5% or higher.
3) Fasting blood glucose level of 126 mg/dL or higher after 8 hours of fasting.
4) Blood glucose level of 200 mg/dL or higher during the 2nd hour of a 75 mg oral glucose tolerance test.

TABLE 1

| Blood glucose level | Fasting blood | Postprandial |
|---|---|---|
| criteria (mg/dL) | glucose | blood glucose |
| Normal | 70~100 mg/dL | Less than 140 mg/dL |
| Prediabetes | 100~125 mg/dL | 140~199 mg/dL |
| Diabetes | 126 mg/dL or higher | 200 mg/dL or higher |

Diabetes must be managed by measuring blood glucose levels using a biosensor and managing the blood glucose levels through diet, exercise, insulin injection, and sulfonylurea medication.

Various biosensors using conventional electrochemistry, optics, and electromagnetic spectroscopy exist. Additionally, numerous glucose biosensors relying on transduction techniques have been reported.

The blood sampling method which directly measures the glucose concentration in the blood is advantageous for the accuracy, but does not unfit for real-time blood glucose check due to intermittent blood collection and raises concerns about secondary infections due to needle use for blood sampling. Therefore, the blood sampling method needs research on non-invasive and continuous blood glucose measurement methods.

Accurate measurement of blood glucose concentration is vital for diabetes treatment. Invasive diabetes detection methods using electrochemical method, metamaterial method, and enzymatic oxidation techniques exist.

Glucose biosensors are used to measure blood glucose levels. Through measurement of glucose levels, diabetic patients can manage their glucose levels effectively with diet, exercise, insulin injections, and sulfonylurea medication, thereby ultimately preventing complications of diabetes such as blindness, chronic kidney failure, heart failure, and nerve damage.

As related conventional art, Korean Patent No. 10-1887602 was disclosed a “Biosensor with RF bandpass structure, method for sensing biological data using same, and method for manufacturing the biosensor”.

FIG. 1 illustrates a conventional biosensor with an RF bandpass structure to measure glucose concentration for diabetes diagnosis.

The biosensor with an RF bandpass structure comprises:
a GaAs substrate used for 9 GHz band high frequency; and
metal lines formed on the substrate.

The metal lines include: a first line and a second line spaced apart from each other at a predetermined distance; at least two diagonal lines connecting the first and second lines using an air-bridge structure; and a third line forming a closed loop with the first and second lines, and the at least two diagonal lines.

The biosensor includes a passivation layer formed between the two or more diagonal lines and a first metal layer of the first and second lines.

The surfaces of the metal lines are coated with gold (Au), and achieve surface roughness of a specific size using etching.

The biosensor with the RF bandpass structure detects changes in the resonant frequency caused by the capacitance of the biosensor, which changes by the permittivity of the material to be measured (serum, D-glucose) located in an air-bridge area, and measures the concentration of the material to be measured based on the changes in the resonant frequency.

Over the past decades, the number of people with diabetes has risen dramatically worldwide, and the need for blood glucose monitoring has increased accordingly (Saeedi et al., 2019; World Health Organization, 2023). Fortunately, glucose detection using biosensing technologies, has witnessed tremendous progress and innovation in the field of biosensing (Corrie et al., 2015; Fiedorova et al., 2022). Real-time invasive glucose level testing has become widely available for diabetic patients (Blicharz et al., 2018; Wang, 2008). Therefore, further developments in glucose detection aim to provide minimally invasive or even non-invasive testing to reduce the pain of diabetic patients, and there is an urgent need for a painless, reliable, and user-friendly glucose sensing system (Kim et al., 2018; Zhang et al., 2021; Zou et al., 2023).

In contrast to attempts to non-invasively measure and extract blood glucose levels, minimally invasive microneedle-based detection system is more stable at current stage (Omar et al., 2023). Microneedle enables detection by extracting and measuring glucose levels in the interstitial fluid, which is virtually painless and the extraction traces disappear in few minutes (Ma and Wu, 2017). Here, the glucose concentration in the ISF has been shown to be highly consistent with the dynamically varying levels in plasma and is reliable, promising to be replaced with the blood puncture of conventional glucose meters (Johnston et al., 2021). Different microneedle-based biosensing systems have been developed, mainly comprising optical and electrochemical platforms (Mohan et al., 2017; Wang et al., 2023; Wu et al., 2022). Ju et al. proposed surface-enhanced Raman spectroscopy (SERS) glucose sensor based on poly (methyl methacrylate) microneedle (PMMA MN) array. The tip of the microneedle is modified with silver particles and the glucose capture agent 1-Decanethiol (1-DT), which displays different Raman spectra for different glucose concentrations under laser, enabling fast and painless blood glucose testing (Ju et al., 2020). Liu et al. proposed an integrated microneedle electrochemical device by microfabrication process, an electroplating process, and an enzyme immobilization. This device detects the H₂O₂ current signal produced enzymatic reaction on the working electrode, highly correlated with signal obtained from a commercial blood glucose meter (Liu et al., 2021). In this field, it has the viability of microneedles as minimally invasive e platforms, capable of supplanting recent conventional invasive detection methods (Tehrani et al., 2022; Xie et al., 2022). Particularly, radio frequency (RF) technology emerges as a promising approach due to its passive detection, miniaturization, and remote sensing capabilities (Dautta et al., 2020; Kim et al., 2015; Qiang et al., 2017). It is not necessary the need for peripheral devices and offers the possibility of non-stimulation, ultra-compact biosensing platforms (Mannoor et al., 2012).

Hydrogel-based microneedles, benefiting from their pronounced swelling properties, can interact intensely with microwave signals during significant morphological changes before and after combing with aim molecules, thereby providing high sensitivity to microwave sensing against reaction (Manzanos et al., 2023; Sridhar and Takahata, 2009; Sun et al., 2023; Turner et al., 2021).

However, the cost of glucose testing for early diabetes diagnosis is increasing, and there has not been provided of a non-invasive, glucose-responsive RF biosensing system within the body's skin in vivo to replace conventional invasive glucose biosensors.

### PATENT DOCUMENTS

Patent Document 1: Korean Registration Patent No. 10-1887602 (granted on Aug. 6, 2018) entitled “Biosensor with RF bandpass structure, method for sensing biological data using the same, and method for manufacturing the biosensor” by Kwangwoon University Industry-Academic Cooperation Foundation

### SUMMARY OF THE INVENTION

Accordingly, the present invention has been made to solve the above-mentioned problems occurring in the prior arts, and it is an objective of the present invention to provide an RF biosensing system using an RF biosensor with microneedles, which can minimally invasively contact the epidermis/dermis/hypodermis of the skin in vivo or the skin surface (on-site)/fat/muscle in vivo by using glucose-responsive hydrogel-based microneedles provided on the bottom of a flexible substrate of an RF biosensor using resonant frequencies within 400˜3,000 MHz, detect and transmit the sensing signal to a reader antenna and a reader antenna sensing circuit through a readout coil by RF biosensing and wireless transmission through EM coupling of RF sensors, and measure the sensing signals by a vector network analyzer (VNA), thereby providing biosensing of proteins, glucose, and fats within the skin in accordance with changes in capacitances of the sensing antenna circuit (LC resonator) of the RF biosensor, resonant frequency shift of the LC resonator, and changes in S-parameters.

To accomplish the above object, according to the present invention, there is provided an RF biosensing system using an RF biosensor with glucose-responsive hydrogel-based microneedles, including: a substrate; a plurality of glucose-responsive hydrogel-based microneedles, which are placed on the bottom of a substrate, and are in minimally invasively contact within the skin in vivo; an RF biosensor, which includes a sensing antenna circuit and a sensing antenna connected to the glucose-responsive hydrogel-based microneedles provided on the bottom of the substrate and including an LC resonator of an RF biosensor provided on the top of the substrate, and a reader antenna and a reader antenna sensing circuit EM coupled within an effective range and connected through a circular readout coil receiving sensing signals by using RF biosensing and wireless transmission; and a vector network analyzer (VNA), which measures changes in capacitances of the sensing signals, central frequency shift of the LC resonator, and changes in S-parameters.

As described above, according to the present invention, the RF biosensing system using the RF biosensor with the microneedles can minimally invasively contact the epidermis/dermis/hypodermis of the skin in vivo or the skin surface (on-site)/fat/muscle in vivo by using glucose-responsive hydrogel-based microneedles provided on the bottom of a flexible substrate of an RF biosensor using resonant frequencies within 400˜3,000 MHz, detect and transmit the sensing signal to a reader antenna and a reader antenna sensing circuit through a readout coil by RF biosensing and wireless transmission through EM coupling of RF sensors, and measure the sensing signals by a vector network analyzer (VNA), thereby providing biosensing of proteins, glucose, and fats within the skin in accordance with changes in capacitances of the sensing antenna circuit (LC resonator) of the RF biosensor, resonant frequency shift of the LC resonator, and changes in S-parameters.

The RF biosensing system using the RF biosensor with the microneedles can measure protein in the skin, blood glucose concentration, and fats in vivo. The RF biosensing system using the RF biosensor with the microneedles is connected to a VNA, and the VNA measures glucose concentration by changes in the resonant frequencies (hypoglycemic frequency f1, normal frequency f2, hyperglycemic frequency f3) of an LC resonator of the RF biosensor and changes in S-parameters (S1, S2, S3), so as to display hypoglycemia, normal, hyperglycemia states, and diagnose diabetes.

### BRIEF DESCRIPTION OF THE DRAWINGS

FIG. 1 illustrates a conventional biosensor with an RF bandpass structure to measure glucose concentration for diabetes diagnosis.

FIG. 2 illustrates an RF biosensor using glucose-responsive hydrogel-based microneedles according to the present invention.

FIG. 3 is a diagram showing the operation of the glucose-responsive hydrogel-based microneedles, a sensing antenna circuit, a sensing antenna, a remotely EM coupled reader antenna, and a reader antenna sensing circuit of FIG. 2.

FIG. 4 is a diagram illustrating a microneedle fabrication process.

FIG. 5 illustrates a biosensing system using an RF biosensor with glucose-responsive hydrogel-based microneedles according to the present invention.

FIG. 6 shows preparation of CMC-pHEA GelMA-ConA hydrogel and fabrication of microneedle-embedded system: (a) synthesis of CMC-pHEA pH-responsive particles and its chemical structure variation; (b) fabrication of microneedles from CMC-pHEA GelMA-ConA glucose-responsive hydrogel; and (c) preparation of embedded system.

FIG. 7 presents material characterization: (A) FTIR spectra; (B) Characteristics of CMC-pHEA GelMA-ConA: (i) CMC-pHEA after freeze-drying; (ii) magnification SEM image; (iii) 1600X temperature strain of CMC-pHEA GelMA-ConA; 1600× magnification SEM image after freeze-drying; (C) Swelling ability of CMC-pHEA GelMA-ConA in different concentration of glucose; and (D) Comparison of shapes and SEM images of hydrogel after swelling: (i) shape comparison; SEM image of hydrogel after immersion in solution of (ii) 0 mM glucose; (iii) 3 mM glucose; and (iv) 20 mM glucose.

FIG. 8 illustrates the characterization of the microneedles: (A) Optical results of the microneedle patch: (i) overall shape; (ii) row of microneedles; and (iii) single microneedle; (B) Extraction capability of the microneedle patch: (i) weight change; (ii) color change; (iii) mechanical characteristics of the displacement-force curve; (C) Reaction of dyed microneedles to insertion-worsened at different glucose concentrations; and (D) Changes in fluorescence intensity: (i) 0 mM; (ii) 3 mM; and (iii) 20 mM.

FIG. 9 illustrates the results of the wireless detection system simulation: (A) Size coupling effect; (B) Different simulation results; (C) Summarized results; (D) Effects of center shift and vertical distance on results; (E) Effect of center relative position on results; (F) Vertical distance effect on results; (H) Swelling condition effect on results; (I) Impact of the degree of swelling; and (J) Frequency shift towards swelling levels.

FIG. 10 illustrates wireless system detection results: (A) Configuration of detection system; (B) Comparison of detection size of microneedle patch; (C) Detection results of comparison between unloaded microneedle patches and loaded microneedle patches; (D) Detection results to different glucose concentrations agarose; (E) Changes in resonant frequency; and (F) Size changes.

FIG. 11 illustrates a real experiment process of CMC-pHEA (FIG. S1).

FIGS. 12 and 13 illustrate a sensing antenna manufacturing process and an HESS simulation (FIG. S2).

FIG. 14 is a photograph of a Readout coil (circular reading coil).

FIG. 15 illustrates a screen showing changes in capacitance of an RF biosensor (LC circuit of LC resonator) with glucose-responsive microneedles→changes in central frequency of the LC resonator→glucose concentration measurement (hypoglycemia/normal/hyperglycemia).

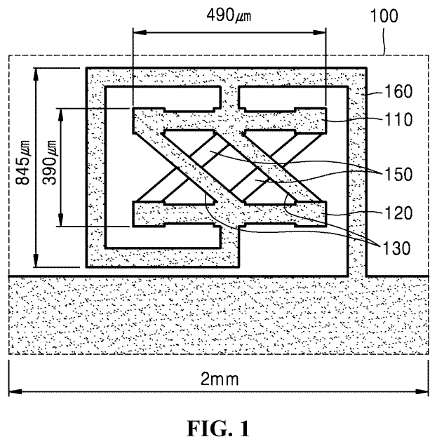

### DETAILED DESCRIPTION OF THE PREFERRED EMBODIMENT

Hereinafter, the configuration and operation according to preferred embodiments of the present invention will be described in detail with reference to the accompanying drawings.

The present invention is not limited to the embodiments disclosed herein but can be realized in various forms by those skilled in the art without departing from the spirit or scope of the present invention. Detailed descriptions of well-known functions or configurations may be omitted when deemed unnecessary to obscure the essence of the invention. The same reference numbers in different figures denote the same or similar components.

The specific embodiments are not intended to limit the invention, but it should be understood that the present invention includes all modifications, equivalents, and replacements belonging to the concept and the technical scope of the invention.

We propose a CMC-pHEA GelMA-ConA hydrogel microneedle-based RF detection platform (Wu et al., 2019). We investigated the interaction between microneedles and RF signals by utilizing dramatic morphological changes of hydrogel microneedles in selective response to glucose, and developed both in-situ and wireless readout platforms equipped with RF biosensors, thereby implementing a minimally invasive, passive, and wireless glucose detection system.

The RF biosensing system using the RF biosensor with the microneedles can minimally invasively contact the epidermis/dermis/hypodermis of the skin in vivo or the skin surface (on-site)/fat/muscle in vivo by using glucose-responsive hydrogel-based microneedles placed on the bottom of a flexible substrate of an RF biosensor using resonant frequencies within 400˜3,000 MHZ, detect and transmit the sensing signal to a reader antenna and a reader antenna sensing circuit through a readout coil by RF biosensing and wireless transmission through EM coupling of RF sensors, and measure the sensing signals by a vector network analyzer (VNA), thereby providing biosensing of proteins, glucose, and fats within the skin in accordance with changes in capacitances of the sensing antenna circuit (LC resonator) of the RF biosensor, resonant frequency shift of the LC resonator, and changes in S-parameters.

Microneedles are used to acquire samples from biological tissues, and are placed on the bottom of a polyimide (PI) flexible substrate in a 15×15 array matrix structure with glucose-responsive hydrogel microneedles. The microneedles have millimeter (mm) diameters to measure glucose concentration by reacting with glucose contained in the body fluid within the skin of a human body.

The substrate uses the polyimide (PI) flexible substrate.

The microneedles are arranged in an n×n array matrix structure on the bottom of the flexible substrate, and the RF biosensor placed on the top of the flexible substrate uses frequencies ranging from 400 to 3,000 MHZ.

Glucose-responsive hydrogels are adhered on the microneedles.

For the glucose-responsive hydrogels, CMC-pHEA GelMA-ConA hydrogels are used.

The penetration depth of the microneedles into the skin is 10 to 100 μm.

The biosensing system having RF biosensors with microneedles can measure proteins, glucose, and fats within the skin in vivo.
1. Antibodies use glucose oxidase as a specific identifier. Glucose reacts with glucose oxidase to produce acid. So, the CMC-pHEA synthesized in a study is highly sensitive to pH, and reacts only to glucose in the swelling state of the hydrogel.
2. To create swelling hydrogel microneedles, a composite material consisting of CMC, KPS, 2-HEA, PEGDA, gelMA, glucose oxidase, and photoinitiators (photo-reactive compounds) is used.
3. For an antenna substrate of the RF biosensor, either an FR4 substrate for a broadband antenna or a polyimide (PI) flexible substrate is used. In addition to the FR4 substrate for the broadband antenna, a ceramic substrate made of ceramic material can be also used.

In an embodiment, the RF biosensor used the polyimide (PI) flexible substrate, and glucose-responsive hydrogel-based microneedles were used on the bottom of the flexible substrate in a 15×15 matrix structure.
4. Since glucose oxidase is used, only glucose oxidase reacts with glucose to produce acid and cause a pH change, thereby having selectivity that selectively responds only to glucose.

Changes in capacitance of an RF biosensor (LC circuit of LC resonator) with glucose-responsive microneedles→changes in central frequency of the LC resonator→glucose concentration measurement (hypoglycemia/normal/hyperglycemia)

Operating frequency of RF biosensor: Frequencies from 400 to 3,000 MHz.

Measurement by vector network analyzer (VNA): S-parameter (S₁₁) experimental results for frequencies from 400 to 3,000 MHz.

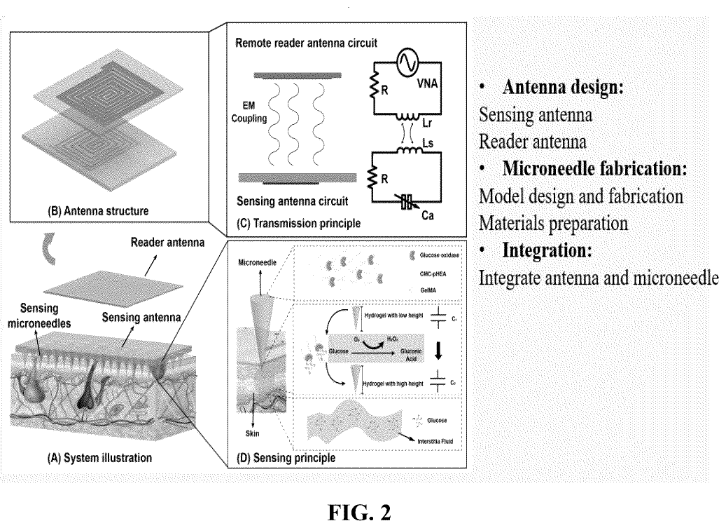

FIG. 2 illustrates an RF biosensor using glucose-responsive hydrogel-based microneedles according to the present invention.

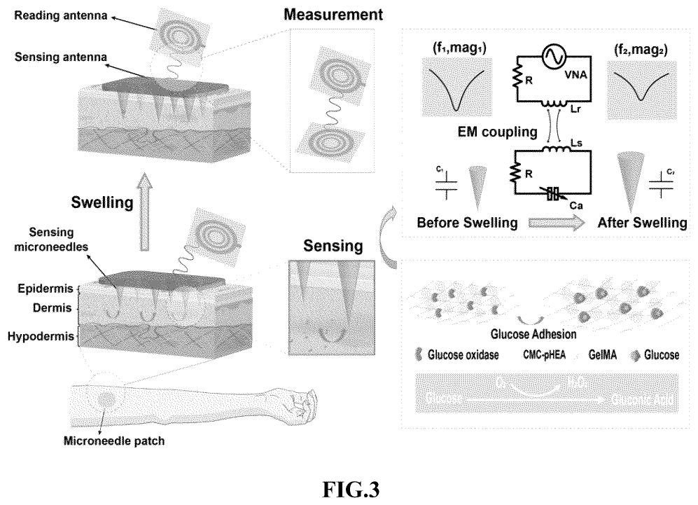

FIG. 3 is a diagram showing the operation of the glucose-responsive hydrogel-based microneedles, a sensing antenna circuit, a sensing antenna, a remotely EM coupled reader antenna, and a reader antenna sensing circuit of FIG. 2.

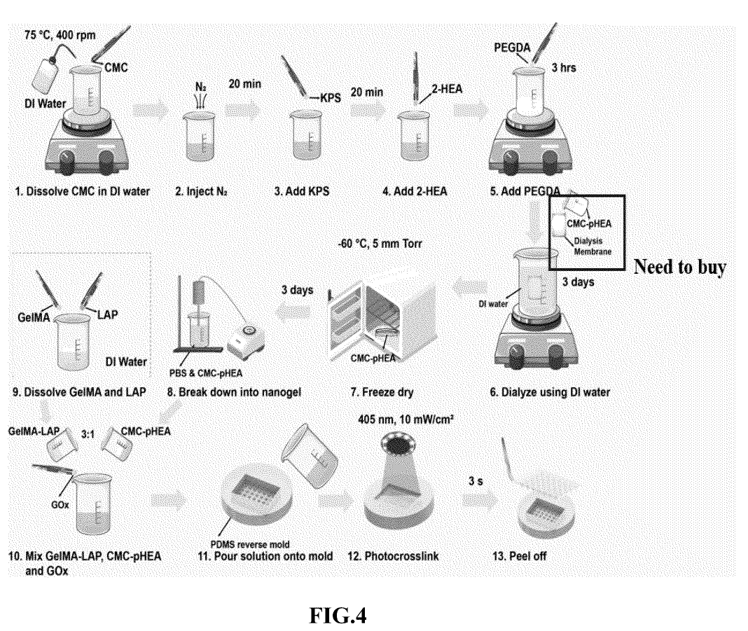

FIG. 4 is a diagram illustrating a microneedle fabrication process.

The RF biosensor is provided on the top of the flexible substrate, and glucose-responsive hydrogel-based microneedles are provided on the bottom of the flexible substrate. The RF biosensor measures changes in capacitances of the RFID wireless biosensor (LC circuit of the RF resonator) and changes in S-parameters in accordance with the glucose concentration of the body fluid (blood) from which antibodies adhered to the ends of the glucose-responsive hydrogel-based microneedles are minimally invasively detected within the skin in vivo.

The biosensing system having the RF biosensor transmits sensing signals wirelessly to the readout coil, the reader antenna, and the reader antenna sensing circuit from by the microneedles on the bottom of the substrate and the sensing antenna of the RF biosensor on the substrate through EM coupling of the sensing antenna and the reader antenna, and measures the sensing signals by the vector network analyzer (VNA).

### Manufacturing of Microneedles

1) DI water & CMC stirring
CMC (carboxymethylcellulose): Water-soluble polymer material (75° C., 400 rpm)
2) N2 gas injection into solution
3) Adding KPS (26° C.)
KPS: potassium peroxodisulfate
4) Adding 2-HEA
2-Hydroxyethyl acrylate (2-HEA) has a hydroxyl (—OH) group, and can improve adhesion due to hydrophilicity
5) Adding PEGDA (biodegradable material)
PEGDA: Polyethylene glycol diacrylate (26° C., 400 rpm)
6) Cooling down the solution
7) Dialyzing for 3 days (Dialysis, 26° C.)
8) Drying using freeze dryer (Freeze dry, −85° C., 500 mm Torr)
9) Powder was swollen in PBS and broken down (1000 rpm, 2 h)
PBS: Phosphate buffered saline
10) Dissolving GelMA and LAP in water
GelMA: Gelatin methacryloyl
LAP: Lithium acylphosphinate salt

Gelatin is obtained by hydrolyzing collagen at high temperatures. GelMA can be produced by dissolving gelatin in phosphate-buffered saline (PBS) (pH=7.4) at 50° C. and reacting it with methacrylic anhydride (MA).
11) Mixing GelMA-LAP solution and CMC-pHEA
12) Adding GOx

Mainly applying glucose oxidase (GOx) on the electrode surface to measure glucose concentration
13) Photocrosslinking under 405 nm

The fabrication of glucose-responsive hydrogel-based microneedles involves mixing the solution at 75° C. to form hydrogel.

The fabrication of glucose-responsive hydrogel-based microneedles involves mixing the solution at 75° C.→dropping hydrogel into the microneedle fabrication container→centrifugation (3500 rpm, 5 min)→Scraping excess→UV crosslinking (40 s)→Peeling off→UV crosslinking (200 s)→dropping resin.

The material of the microneedle is GelMA (gelatin methacryloyl) hydrogel. Hydrogel is a hydrophilic polymer network with a high water content matrix. GelMA hydrogel, based on gelatin, is biocompatible and biodegradable. The swelling ratio of the hydrogel is measured in pH 7.4 and pH 2.5 PBS buffer solutions at room temperature (23° C.).

$$Swelling ratio ( \% ) {=}^{\frac{{W}_{s} + {W}_{A}}{{W}_{d}} \times 100} ,$$

wherein Ws is the weight of the swollen hydrogel (g), and Wd is the weight (g) of the dried hydrogel.

Hydrogel is a hydrophilic polymer material with a three-dimensional network structure that does not dissolve in water but swells, and can contain a large amount of water. After swelling in an aqueous solution, the hydrogel is widely used in the field of pharmaceuticals due to thermodynamical stability and unique hydrophilicity and flexibility.

### [Preparation Materials]

Carboxymethyl cellulose,
2-Hydroxyethyl acrylate,
Potassium persulfate.

### Embodiment

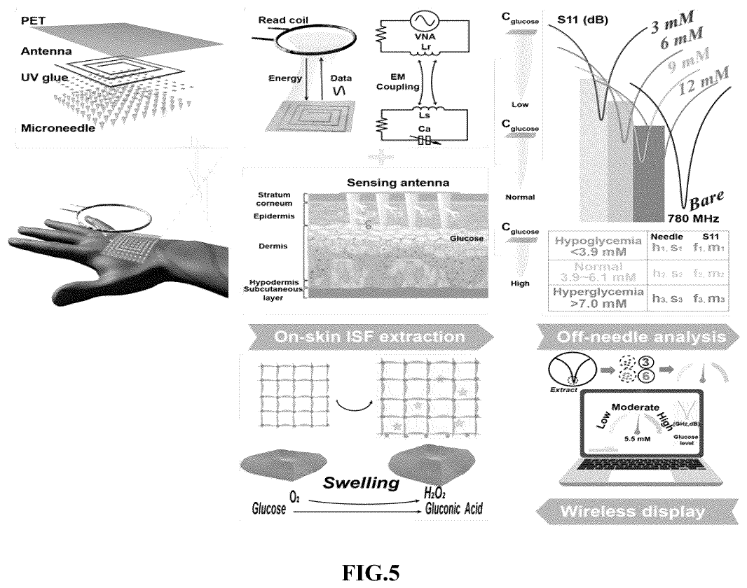

FIG. 5 illustrates a biosensing system using an RF biosensor with glucose-responsive hydrogel-based microneedles according to the present invention.

The biosensing system using the RF biosensor with glucose-responsive hydrogel-based microneedles includes: a substrate; a plurality of glucose-responsive hydrogel-based microneedles, which are formed in an n×n matrix structure (where n≥1 is a natural number) on the bottom of a substrate, and minimally invasively contact the epidermis, dermis, hypodermis within the skin in vivo, or the skin/fat/muscle in vivo; an RF biosensor, which includes a sensing antenna circuit and a sensing antenna connected to the glucose-responsive hydrogel-based microneedles and including an LC resonator of an RF biosensor provided on the top of the substrate, and a reader antenna and a reader antenna sensing circuit EM coupled within the effective range of 5 mm and connected through a circular readout coil receiving sensing signals by using RF biosensing and wireless transmission; and a vector network analyzer (VNA), which measures changes in capacitances of the sensing signals of the RF biosensor, central frequency shift of the LC resonator, and changes in S-parameters.

The plurality of microneedles formed in a matrix structure is provided on the bottom of the substrate, and are used to contact minimally invasively on the epidermis, dermis, hypodermis of the skin, or skin/fat/muscle in vivo.

The vector network analyzer (VNA) includes the plurality of microneedles formed in an n×n array matrix structure on the bottom of the substrate, and is used to contact minimally invasively on the epidermis, dermis, hypodermis of the skin, or skin/fat/muscle in vivo.

The vector network analyzer (VNA) is used for RF and Microwave measurements, and contacts minimally invasively into the skin in vivo. When measuring a sample (blood) of the skin, the vector network analyzer (VNA) connected to the readout coil receiving the sensing signals from the sensing circuit of the sensing antenna circuit (LC resonator circuit) of the RF biosensor by wireless detection measures the glucose concentration in the body fluid in accordance with changes in capacitances of the sensing signals of the RF biosensor, central frequency shift of the LC resonator, and changes in S-parameters.

The substrate can be either an FR4 substrate, a flexible substrate, or a ceramic substrate for a broadband antenna for an antenna system of the RF biosensor.

In an embodiment, the substrate uses the flexible substrate, which can be either a PET substrate or a polyimide (PI) substrate.

The glucose-responsive hydrogel-based microneedles of a 15×15 array matrix structure is provided on the bottom of the flexible substrate, the RF biosensor to which the sensing antenna and the sensing antenna circuit are attached is provided on the top of the flexible substrate, and the VNA is connected above the RF sensor via the readout coil. A patch antenna is attached to the skin, and the readout coil provided above senses the patch antenna within the effective range of 1 to 5 mm to measure resonant frequencies.

In an embodiment, the sensing antenna of the RF biosensor is a patch antenna with a radiator provided at the center and metal lines of square-shaped spiral structure in an N-turn structure. The metal lines are made of gold (Au), silver (Ag), or copper (Cu), have line width W=0.1˜1 mm and line spacing S=0.1˜1 mm, and are spaced apart at regular intervals. In an embodiment, the metal lines have a line width W=0.5 mm and line spacing S=0.5 mm.

In the biosensing system having the RF biosensor, the sensing antenna and the reader antenna of the RF biosensor provided on the top of the substrate use RF frequencies in the range of 400 to 3,000 MHz.

The biosensing system using the RF biosensor with microneedles includes glucose-responsive hydrogel-based microneedles and sensing antenna circuits, and sensing antennas which, through EM coupling, transmit sensing signals within an effective range of 5 mm or less by using RF biosensing and wireless transmission/detection techniques; a reader antenna and a reader antenna sensing circuit received these signals through the circular Readout coil, connected to a VNA.

When the RF biosensor with microneedles is in contact with the skin in vivo and detects glucose in the skin or capillaries and the interstitial fluid (ISF) in subcutaneous fat, complex permittivity and capacitance (C) at different glucose concentrations are changed, the changes alter and move the resonant frequencies of the LC resonator of the sensing antenna circuit of the RF biosensor, and S₁₁ is also changed, thereby detecting accurate glucose concentration through measurement of the resonant frequencies.

The complex permittivity of glucose varies with glucose concentration, and it manifests as changes in the capacitance (C) of the RF biosensor. So, the complex permittivity and capacitance (C) in accordance with different glucose concentrations are changed, thereby detecting glucose concentration by measuring resonant frequencies of the sensing antenna circuit of the RF biosensor.

The RF biosensor uses the microneedles made from glucose-responsive hydrogels placed on the bottom of the flexible substrate to minimally invasively extract the interstitial fluid (ISF) in subcutaneous fat without any pain. The interstitial fluid (ISF) is the fluid between cells, outside the capillaries in the skin, so measures the glucose level slightly later than the glucose level in the blood vessels.

In an embodiment, when the RF biosensing system minimally invasively contacts the skin composed of epidermis, dermis, hypodermis, or skin/muscle/fat in vivo to measure a body fluid sample (blood), the vector network analyzer (VNA) receives sensing signals through EM coupling and wireless detection in the swelling state from the sensing antenna circuit (LC resonator) and the sensing antenna of the RF biosensor (LC resonator), and transmits the signals to the circular Readout coil, the reader antenna, and the reader antenna sensing circuit. The VNA connected to the readout coil measures the glucose concentration in accordance with changes in capacitances of the sensing signals of the RF biosensor, central frequency shift of the LC resonator, and changes in S-parameters.

The RF biosensing system, which minimally invasively contacts the skin in vivo to measure a body fluid sample (blood), includes an RF biosensor, which includes glucose-responsive hydrogel-based microneedles on the bottom of the flexible substrate, a sensing antenna circuit (LC resonator) and a sensing antenna of the RF biosensor on the substrate, and a sensor unit remotely receiving (wireless transmission) sensing signals by using RF biosensing and wireless transmission remotely EM-coupled within the effective range of 1 to 5 mm and having a reader antenna and a reader antenna sensing circuit through a circular readout coil remotely EM-coupled within the effective range of 5 mm, wherein the readout coil is connected to a vector network analyzer (VNA).

The RF biosensor with glucose-responsive hydrogel-based microneedles, when minimally invasively contacting the skin in vivo to measure the skin, detects sensing signals through the readout coil within the effective range of 1 to 5 mm, and the VNA connected to the readout coil, the reader antenna, and the reader antenna sensing circuit (wireless detection) measures the glucose concentration in the skin and capillaries in accordance with changes in capacitances of the sensing signals of the RF biosensor, central frequency shift of the LC resonator, and changes in S-parameters.

The RF biosensor using the glucose-responsive hydrogel-based microneedles can measure the glucose concentration by using capacitance and permittivity of the RF biosensor changing in accordance with the glucose concentration in the skin or capillaries. The overall impedance of the RF biosensor varies depending on the electrical properties (permittivity, capacitance) of the substance being measured (e.g., glucose).

In measurement in skin, since different glucose concentrations is related to have different permittivities, the capacitance of the LC resonator of the sensing antenna circuit of the RF biosensor is changed and the resonant frequencies (fₒ) of the LC resonator are moved differently. Thus, changes in

$${f}_{o} = \frac{1}{2 π \sqrt{LC}} ,$$

namely, electrical characteristics (S_11) of the LC resonator of the RF biosensor during skin measurements is measured.

The complex permittivity of glucose depends on the glucose concentration, hence different electrical resonances occur in the RF biosensor (LC resonator) depending on the glucose concentration in the skin or capillaries. Changes in permittivity for different glucose concentrations manifest as differences in the capacitance (C) of the RF biosensor (LC circuit of the RF biosensor).

In another embodiment, the RF biosensing system using an RF biosensor with glucose-responsive hydrogel-based microneedles can be manufactured as an RF glucometer. In this case, the RF glucometer (IoT device) includes a sensor unit connected to a control unit (MCU), which is connected to a storage part and a display part. The RF glucometer may also include a communication unit providing Bluetooth or Wi-Fi communication. The RF glucometer transmits and stores glucose concentration to a diabetes management server via wired or wireless networks, and displays the measured glucose concentration of body fluids (blood, saliva) on a diabetes measurement application on a computer, a smartphone, or a tablet PC and displays hypoglycemia/normal/hyperglycemia on the computer screen.

The RF biosensing system equipped with the RF biosensor, for example, the RF glucometer, when being used minimally invasively into the skin in vivo using microneedles provided on the bottom of the flexible substrate of the RF biosensor, may comprise: a control unit (MCU), which receives sensing signals from a sensing antenna circuit and a sensing antenna of the RF biosensor to a readout coil, a reader antenna and a reader antenna sensing circuit through EM coupling and wireless detection to measure the glucose concentration in accordance with changes in capacitances of the sensing signals, shifts and changes (fc) in central frequencies of the LC resonator; a storage unit connected to the control unit (MCU); a display unit connected to the control unit (MCU); and a communication unit connected to the control unit (MCU) to provide Bluetooth or Wi-Fi communication.

### 2. Materials and Methods

### 2.1 Reagents and Instruments

i) Materials for the synthesis of CMC-pHEA pH-responsive particles: sodium carboxymethyl cellulose (CMC, Mw˜90,000), potassium persulfate (KPS), and 2-hydroxyethyl acrylate (2-HEA)
ii) Materials for further synthesis of glucose-responsive hydrogels: GelMA (DS90) and LAP
iii) Glucose oxidase (GOx) and concanavalin A (ConA)
iv) Materials for artificial agarose skin model: agarose

### *Materials and Instruments for Synthesis of CMC-pHEA GelMA-ConA

Microneedles: freeze dryer (ilShinBioBase, FD8512), cell disruption equipment (SONICS & MATERIALS, VC505), UV equipment (Kugou) and centrifuge (LABOGENE, 2236R) were used. Dialysis bags and UV resin were purchased from Beijing Yikang Prosperous Biotechnology Co., Ltd. and September Optoelectronics Technology Co. respectively.

Characterization instruments: Fourier-transform infrared spectroscopy (FTIR) equipment (SS), scanning electron microscope (SEM) equipment (JOEL JSM-7001F), universal testing machine (MTS, CMT6103), and optical microscope (OLYMPUS, SZX16) were used.

### 2.2 Preparation of CMC-pHEA pH-Responsive Particles

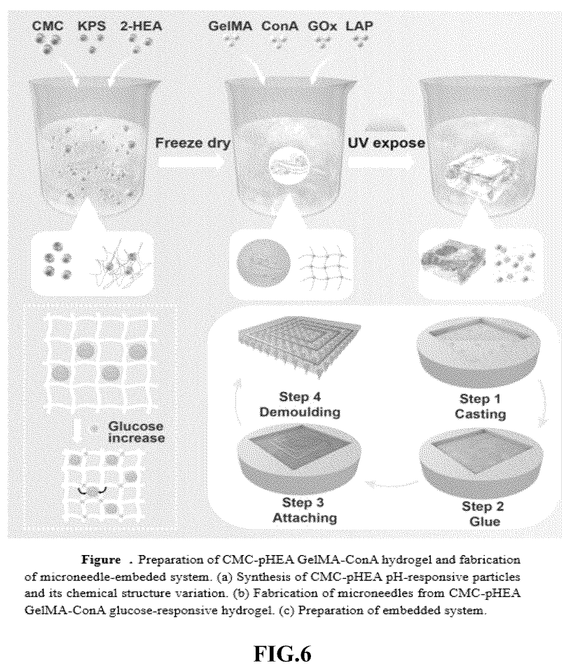

The preparation process of CMC-pHEA pH-responsive particles is illustrated in FIG. 6. First, 1.2 g of CMC was added to DI water (ratio CMC:DI=1.2 g:100 mL), and placed on a hot plate (75° C., 400 rpm) to dissolve it fully, then it was left in a vacuum atmosphere for 20 minutes to prevent oxidation. Thereafter, 0.2 g of KPS was added to the mixture, and reacted for 20 minutes at room temperature. Thereafter, 18 mL of 2-HEA and 1 mL of PEGDA were added to the CMC and KPS mixture respectively, and reacted for three hours at 400 rpm at room temperature. It is not too high a temperature to prevent thermal cross-linking. When the reaction was complete, the mixture was poured into a dialysis bag, sealed, and then, placed in DI water for three days, changing DI water once. Thereafter, the mixture was refrigerated at −80° C. and freeze-dried in a freeze dryer (−85° C., 5 mTorr) for three days. The lyophilised CMC-pHEA samples were weighed and mixed with six times the weight of PBS solution, and then, crushed under a cell crusher for ten minutes into a viscous gel-like liquid. Unused lyophilised samples were stored at −20° C. and the gelatinous liquid was stored at 4° C. It can be seen that after the addition of KPS to the CMC mixture, H of the —OH group on the chemical structure of the CMC is replaced in the CMC-KPS mixture. Thus, upon addition of 2-HEA in the CMC-KPS mixture, 2-HEA molecules bind to the remaining-O-group. After the addition of PEGDA, the linkage of the CMC-2-HEA molecules is connected to the other CMC-2-HEA molecules via the PEGDA molecules and a pH-responsive network is formed.

FIG. 6 shows preparation of CMC-pHEA GelMA-ConA hydrogel and fabrication of microneedle-embedded system: (a) synthesis of CMC-pHEA pH-responsive particles and its chemical structure variation; (b) fabrication of microneedles from CMC-pHEA GelMA-ConA glucose-responsive hydrogel; and (c) preparation of embedded system.

### 2.3 Fabrication of Microneedles from CMC-pHEA GelMA-ConA Glucose-Responsive Hydrogel

To manufacture glucose-responsive microneedles, the prepared CMC-pHEA pH-responsive gel was first mixed with GelMA (10% of the total mixture), LAP (5% of the total mixture), GOx (32 mg/mL), and ConA (32 mg/mL) to synthesis a CMC-pHEA gelMA-ConA hydrogel solution, which was stored at 4° C. as illustrated. The liquid has a low-temperature coagulation characteristic, while excessive heat can inactivate the enzyme, so it needs to be kept in a water bath with heat at approximately 50° C. before use.

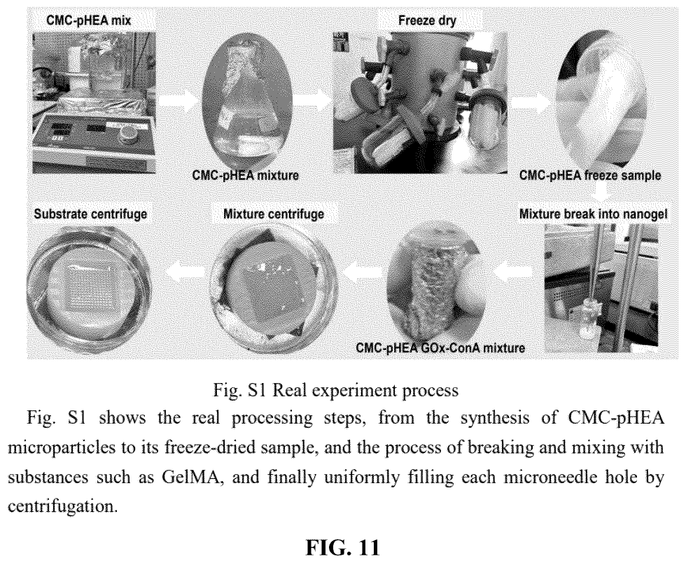

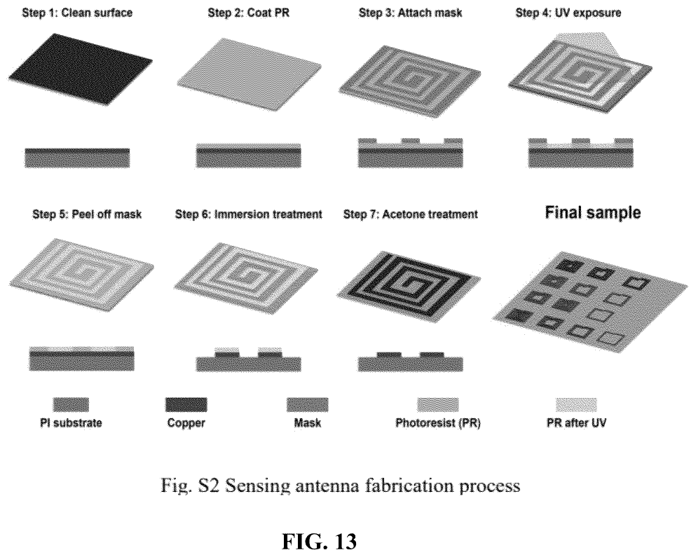

In the microneedle sensing patch fabrication process, the mixed hydrogel solution (250 μl) was dropped onto the microneedle mold and then placed in a centrifuge for five minutes (3500 rpm, 25° C.). After the gel had slightly solidified, the hydrogel was scraped off the surface of the microneedle pores with a spatula and exposed to UV light for 40 s. It was then left to dry for approximately two hours in a ventilated area at room temperature. Afterward, adequate resin was applied to sensing antenna surface and microneedle mold surface through brush, followed by closely attaching and irradiated with UV light for 40 s to ensure a secure connection between the microneedle and the antenna. After drying for one hour in a ventilated area at room temperature, the microneedles are peeled off with tweezers and as much force as possible perpendicular to the microneedles, stored in a cool, dry place and used within a week. Moreover, the real materials synthesis and flexible antenna fabrication process are illustrated in FIG. 11 (FIG. S1) and FIG. 13 (FIG. S2).

FIG. 11 illustrates a real experiment process of CMC-pHEA (FIG. S1).

FIG. S1 illustrates the real process steps from the synthesis of CMC-pHEA micro-particles to freeze-drying samples, including breaking and mixing materials such as GelMA and uniformly filling each microneedle hole through centrifugation.

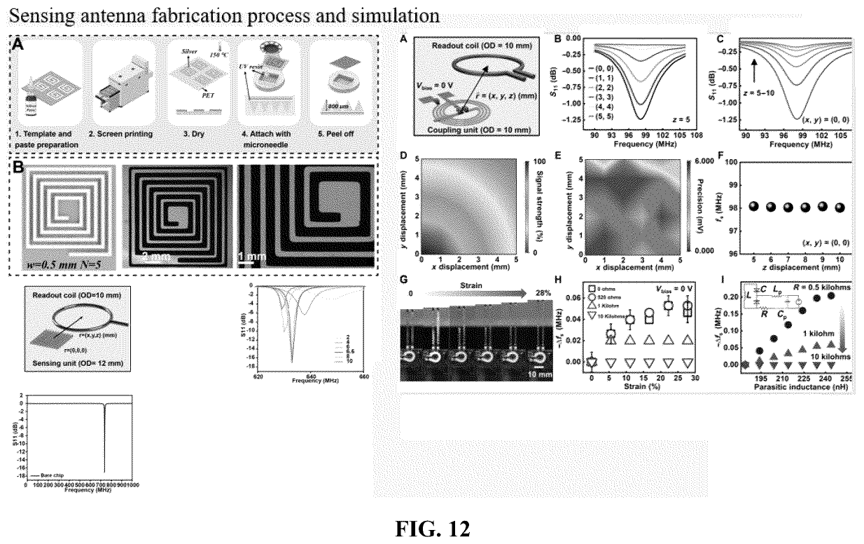

FIGS. 12 and 13 represent the manufacturing process of the sensing antenna and HFSS simulation (FIG. S2). To manufacture a flexible antenna used as the sensing antenna of the RF biosensor, the surface of the polyimide (PI) substrate was first thoroughly cleaned to make it smooth and clean. Copper was coated onto the surface of the PI substrate, and then, photoresist (PR) was coated thereon. Thereafter, a printed mask on which a specific circuit pattern was printed was attached to the surface of the PI substrate, and then was exposed to 405 nm UV light for 40 seconds. After UV exposure, the mask was removed, and the substrate was immersed in a solution. Once the substrate had fully reacted, it was treated with acetone to produce the flexible antenna.

The actual sensing antenna of the RF biosensor is a square-shaped spiral antenna with an N-turn structure made of gold (Au) or copper (Cu). The metal lines were produced with a line width (W) of 0.5 mm and a line spacing(S) of 0.5 mm.

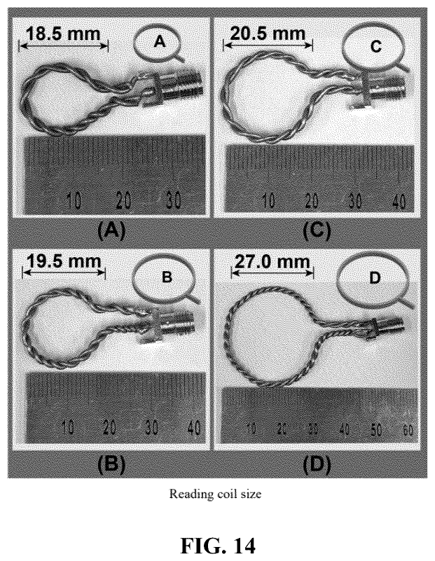

Referring to FIG. 14, the readout coil is a circular reading coil, and was tested while increasing diameters of 18.5 mm, 19.5 mm, 20.5 mm, and 27 mm.

### 2.4 Characterization of CMC-pHEA GelMA-ConA Glucose-Responsive Hydrogel

### 2.4.1 Fourier Transform Infrared (FT-IR) Analysis

The structures of CMC-pHEA pH-responsive gel and CMC-pHEA GelMA-ConA hydrogels were characterized by FT-IR absorption spectroscopy using with a resolution of in the range from 4000 to 400 cm⁻¹.

### 2.4.2 Morphology Characterization

To observe the structure of CMC-pHEA pH-responsive lyophilized samples, SEM was used to observe its inner structure at lyophilized conditions. Also, in order to observe the internal structure of the UV cross-linked glucose-responsive hydrogels, the gelled samples were firstly treated with liquid nitrogen for five minutes, left to freeze-dry for two days, and subsequently the internal porous structure was observed by SEM.

### 2.4.3 Analysis of Swelling Ability Characteristics of Hydrogel

CMC-pHEA GelMA-ConA glucose-responsive hydrogel was dropped in equal amounts onto multiple 3D printed mold of the same design and after the same time UV treatment, weighed and recorded. The UV cross-linked samples were then soaked in different concentrations of glucose solutions for 10 minutes, surface water was wiped, then weighed and recorded. Furthermore, the soaked hydrogel samples were also treated with liquid nitrogen and freeze-dried, followed by using SEM to observe the internal structure differences.

### 2.5 Characterization of Hydrogel Microneedle

### 2.5.1 Optical Results of Microneedle

In order to observe the surface condition of microneedles, the optical microscope, phone camera, and SEM were used to obtain overall microneedle fabrication, and detailed height. Especially, we specially mixed a little Rhodamine B into the hydrogel to make the microneedle more visual.

### 2.5.2 Swelling and Extraction Ability

To examine the swelling properties and in vitro extraction capacity of microneedles, an agarose model of simulated artificial skin containing glucose was made (Supporting information). On the one hand, conventional quality characterization was used to compare the quality change before and after microneedle insertion, and an optical microscope was used to record the height variation before and after microneedle insertion. To visualize the extraction capacity, transparent microneedles were inserted into the agarose model containing Rhodamine B to observe whether the microneedles were stained.

### 2.5.3 Mechanic Property Test

The mechanical property test was performed by a universal testing machine. By applying different levels of vertical forces, the deformation of the microneedle was observed and recorded.

The insertion ability of the microneedles was initially obtained by inserting them into the agarose model, and then, holes left behind were observed. In an experiment, the microneedles were inserted into the pig skin and the holes left behind were observed.

The plurality of microneedles were placed in a room-temperature unsealed, room-temperature relatively sealed, low-temperature unsealed, and low-temperature sealed environment, and the morphological changes of each microneedle were observed and recorded daily with a microscope to determine its storage stability.

### 3. Results

### 3.1 Characterization of CMC-pHEA and CMC-pHEA GelMA-ConA Hydrogel Particles

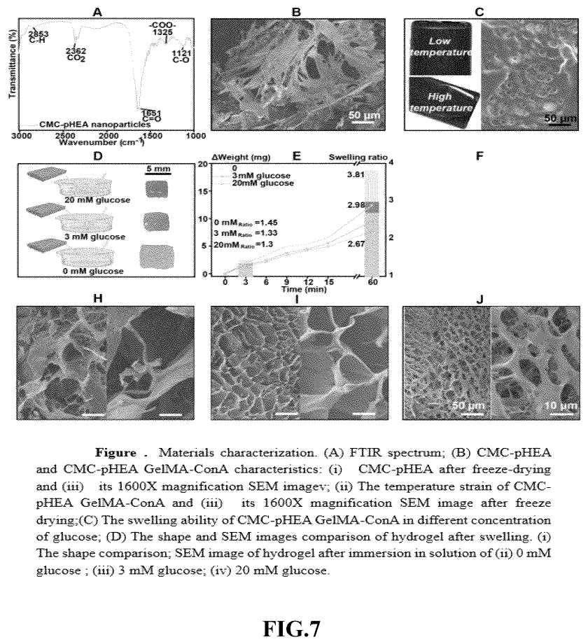

FT-IR spectral results proved that both the cross-linking of CMC-pHEA by freeze-drying was successful, as illustrated in FIG. 7A. In the spectrum, peak at 1651 cm⁻¹ corresponding to C═O double bond proved that CMC molecules were highly crosslinked during synthesis process. In addition, the peak at 2853 cm⁻¹ represented symmetric stretching vibrations of methylene C—H, that at 1325 cm⁻¹ was caused by the symmetric stretching vibration band of carboxylate (—COO—) in CMC. Peak of lower frequency at 1121 cm⁻¹ in the hydrogel spectra represented the stretching vibrations of the ester C—O. While the peak at 2362 cm⁻¹ originated from the carbon dioxide dissolving in solution from air and is not related to sample synthesis. Based on the above results, the crosslinking of the CMC-pHEA particles was confirmed successfully (Park et al., 2018) (Schultz et al., 1996). FIG. 7B illustrates the morphology characterization of freeze-dried CMC-pHEA nanoparticles through 300× SEM magnification of showing an internal filamentary mesh structure. The synthesized un-crosslinked CMC-pHEA GelMA-ConA hydrogel maintains strong temperature rheology (FIG. 7C), i.e., low-temperature solidification and high-temperature melting. The 300× SEM magnification of dried crosslinked sample shows the internal structure of small pores. Furthermore, FIGS. 7D and 7E validates the glucose responsiveness of the UV-crosslinked CMC-pHEA GelMA-ConA hydrogel. Their morphologies obtained by a phone camera are significantly different as shown in FIG. 7D. The swelling growth rates of hydrogels with almost identical initial weights were significantly different in different concentrations of glucose solutions, and the swelling ratios of hydrogels in 0 mM, 3 mM, and 20 mM are 3.81, 2.98, and 2.674 respectively at 60 min. The results indicate that the swelling rates decreased with increasing glucose concentrations and CMC-pHEA GelMA-ConA responded significantly to glucose. Also, the biocompatibility is validated in FIG. 7F, which proved the hydrogel is non-toxicity when attaching human body. In addition, the internal microstructure was observed by SEM, and it could be seen that dense porous structures were formed inside all the soaked hydrogels compared to the unswollen hydrogels. Among them, the porous size of the hydrogels immersed in water (FIG. 7H) was significantly larger than that of the 3 mM glucose solution (FIG. 7I), which in turn was larger than that of the 20 mM glucose solution (FIG. 7J). This indicates that benefiting from the construction of the internal network of CMC-pHEA GelMA-ConA hydrogels, the hydrogel can bind to glucose molecules and cause different swelling of the internal network. Moreover, the UV exposure time effect on the responsibility needs appropriate cross-linking time.

FIG. 7 presents material characterization: (A) FTIR spectrum; (B) Characteristics of CMC-pHEA GelMA-ConA: (i) CMC-pHEA after freeze-drying; (ii) 1600× magnification SEM image; (iii) temperature strain of CMC-pHEA GelMA-ConA; 1600× magnification SEM image after freeze-drying; (C) Swelling ability of CMC-pHEA GelMA-ConA in different concentration of glucose; and (D) Comparison of shapes and SEM images of hydrogel after swelling: (i) shape comparison; SEM image of hydrogel after immersion in solution of (ii) 0 mM glucose; (iii) 3 mM glucose; and (iv) 20 mM glucose.

### 3.2 Characterization of CMC-pHEA GelMA-ConA Microneedles

To verify the fabrication of the microneedles, the microneedle hydrogel was added color-dye particles to fabricate microneedles, and then, the microneedle patch was first photographed with an optical microscope and camera.

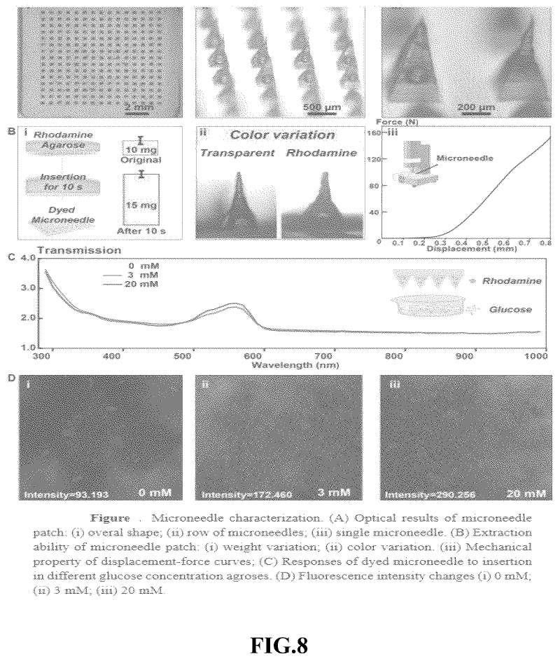

The microneedle patch is completely formed in a 15*15 array. Measurement of a particular row of microneedles, as shown in FIGS. 8A (ii) and 8A (iii), shows that both microneedle height and needle morphology are highly uniform. The extraction ability of the microneedles was verified by inserting them into agarose containing Rhodamine and observing the microneedle needles before and after insertion. The weight change was further quantified and it can be seen that the microneedle patch can rapidly extract ISF, with an outside ISF of about 5 mg in 10 s when the weight is only 10 mg.

As shown in FIGS. 8B (i) and 8B (ii), it also can be seen that the microneedle needles before insertion were clear and sharp, and the needles after insertion were stained and slightly swollen. Similarly, the mechanical properties of the microneedle and the insertion capability are important for the ability to insert into the skin and extract ISF. Universal testing machines are used to obtain accurate force-displacement curves. The displacement increased as the force increased. The relationship between force and compression was obtained and exhibited in FIG. 8B (iii). MNs withstand 10 N force at 300 μm displacement, which could effectively penetrate the skin surface. To further verify the swelling response of microneedles to different glucose concentrations, Rhodamine was incorporated into the hydrogel and microneedles were prepared, inserted into agarose with different glucose concentrations, and their fluorescence intensity was measured. As illustrated in FIG. 8C, the transmission rates of microneedle inserting in different concentration agarose are different and regular, the fluorescent intensities increase from 93.193, 172.460, 290.256 with the increase of glucose concentration, thereby decreasing the swelling ratio (FIGS. 8D to 8F).

FIG. 8 illustrates the characterization of the microneedles: (A) Optical results of the microneedle patch: (i) overall shape; (ii) row of microneedles; and (iii) single microneedle; (B) Extraction ability of the microneedle patch: (i) weight variation; (ii) color variation; (iii) mechanical property of the displacement-force curve; (C) Responses of dyed microneedles to insertion in different glucose concentratin agarose-worsened at different glucose concentrations; and (D) Changes in fluorescence intensity: (i) 0 mM; (ii) 3 mM; and (iii) 20 mM.

### 3.3 Wireless Detection System Simulation and Construction

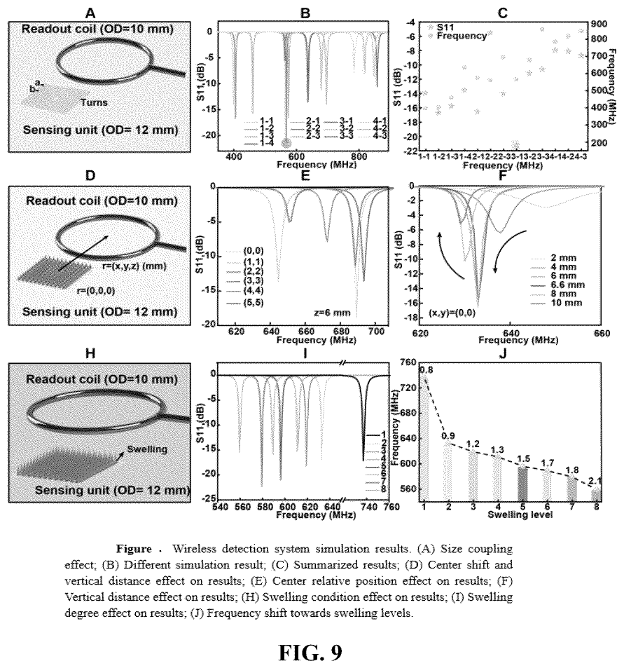

FIG. 9 illustrates the results of the wireless detection system simulation: (A) Size coupling effect; (B) Different simulation results; (C) Summarized results; (D) Center shift and vertical distance effect on results; (E) Center relative position effect on results; (F) Vertical distance effect on results; (H) Swelling condition effect on results; (I) Swelling degree effect on results; and (J) Frequency shift towards swelling levels.

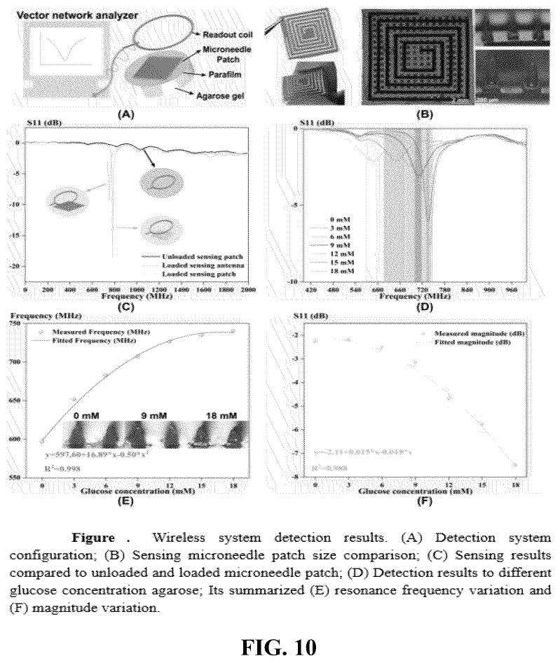

To determine the optimal sensing system and verify the sensing effect, the biosensing system having the RF biosensor was first simulated using HFSS simulation (S₁₁ parameter according to frequencies). Considering the overall size of the microneedle (12 mm*12 mm), the overall size of the sensing antenna was designed by advanced design software (ADS) which is RF design software and was set to 15*15 mm, as shown in FIG. 9A, and the number of turns, line width and line spacing were optimized and adjusted. Simulation results are shown in FIGS. 10B and 10C, which illustrates the antenna with structure 3-1 showing the sharpest S₁₁ peak of −22 dB at 580 MHz. The antenna with structure 3-1 has the sharpest S₁₁ peak of −22 dB at 580 MHz.

To construct a highly-sensitive and effective wireless detection system, exploratory experiments were performed on several factors that strongly influence on the wireless detection system results as shown in FIG. 9. Considering the size of the microneedle patch, the overall size of the sensing antenna is designed consistently as 12 mm*12 mm, while the wire width, number of turns and coils of the spiral coil are determined as different design factors. Moreover, the reading coil sizes are also considered and explored, all experiments are executed at a fixed distance of 3 mm with the size shown in FIG. 9A. FIG. 9B illustrates the results of a coil-A, a coil-B, a coil-C, and a coil-D responding to spiral-sensing antennas, with insets of magnitude variation summary. In analysis, the dimension of the reading coil exerted a pronounced influence on the sensing outcomes. Factors such as the number of turns, wire width, and spacing of the spiral line exhibited irregular effects on the results. This imperative of preliminary underscores the experimentation in optimizing wireless sensing systems. In a side-by-side comparison, the amplitude response of the coil-C consistently outperformed the other three groups. Further, FIG. 9C illustrates the vertical distance effect on the sensing results of the optimum selected group. Due to the presence of sensing antennas, peaks generated by inductively coupling appear distribute between 650 MHz to 700 MHz. In FIG. 9D, as the distance between two antennas increases (from 0 mm to 9 mm) gradually, the intensity of the peak appears to increase first from −7 dB to −45 dB from 0 to 4 mm, then decreased to −6 dB to 9 mm. The results indicated that the optimum distance for detection was around 2.5 mm. The measured original frequency value can be used as a calibration before sensing.

### 3.4 Wireless Detection Results

Based on the above prerequisite optimal detection conditions, including antenna size and morphology, distance and location (fixed at vertical 2.5 mm), the glucose detection capability of the wireless system was measured and investigated. The biosensing system to measure the glucose concentration in accordance with changes in capacitance and changes in resonant frequencies of the LC resonator of the sensing antenna circuit of the RF biosensor using the vector network analyzer (VNA) is illustrated in FIG. 10(A). FIG. 10(B) illustrates a flexible sensing patch incorporating integrated microneedle and antenna. The dimensions of this flexible sensing patch are 15 mm×15 mm, and shows its adaptability to bending and microneedle size compared to wire size.

FIG. 10 illustrates wireless system detection results: (A) Configuration of detection system; (B) Comparison of detection size of microneedle patch; (C) Detection results of comparison between unloaded microneedle patches and loaded microneedle patches; (D) Detection results for agarose with different glucose concentrations; (E) Changes in resonant frequency; and (F) Size changes.

First, the results of the system having microneedles and a basic antenna patch are represented in FIG. 10C. Compared to the unloaded condition, resonance appears due to the added sensing antenna but is slightly reduced by the microneedle's adhesion. FIG. 10D shows S₁₁ spectrum responses on various glucose concentrations in agar over 10-second intervals, and FIG. S10 shows other results. As the glucose concentration goes up, resonance gradually moves to a higher frequency, and the magnitude also increases. In FIG. 10E, the resonance frequency is summarized, changing from 600 MHz at 0 mM to 748 MHz at 18 mM, with fitted curves following the quadratic function y=597.6+16.89x−0.5x² and COD of 0.998. Similarly, FIG. 10F shows the magnitude changes, shifting from −2.1 dB to −7.5 dB, following the quadratic function y=2.11+0.015x−0.018x² and COD of 0.988. These results indicate that the glucose-sensitive microneedle is closely related to glucose concentration, significantly affecting the antenna parameters. Various glucose microneedle detection platforms are summarized in the following table. It can be seen that different types of microneedles have been developed and applied to detection methods such as electrochemistry and colorimetry based on different detection principles. We first proposed a combined hydrogel microneedle swelling and radiofrequency detection methods achieve highly sensitive detection of glucose.

Table 2 shows comparison of different microneedle sensors.

TABLE 2

| | | | Recognition | Detection | | |
|---|---|---|---|---|---|---|
| Reference | Analytes | Microneedle types | elements | approach | Principle | Sensitivity |
| (Liu et | Glucose | Ag/AgCl deposited | GOx | electrochemical | Different current produced when | 0.0416 uA/ |
| al., 2021) | | Prussian blue/Solid | | | GOx reacts with glucose | mM |
| (He et al., | Glucose | Hyaluronic acid (HA)/ | GOx | colorimetric | GOx reacts with glucose produces | 0.0125 Hue/ |
| 2021) | | Hydrogel | | | gluconic acid generates pH changes | mM |
| | | | | | with color variation | |
| (Lu et al., | Glucose | photonic crystals/ | | spectrum | Hydrogel swelling when PBA | 30 nm shift/ |
| 2023) | | Hydrogel | | analyzer | combines with glucose and generates | mg/mL |
| | | | | | color variation | |
| (Zheng et | Glucose | Silk fibers/Hydrogel | GOx | electrochemical | Different current produced by H2O2 | 75 nA/ |
| al., 2022) | | | | | when GOx reacts with glucose | mM |
| (ZHANG | Glucose | photonic crystals | PBA | colorimetric | Hydrogel volume changes when | 25 nm/ |
| et al., 2022) | | PBA/Hydrogel | | | exposure to glucose and generates | mM |
| | | | | | optical wavelength vary | |
| (Parrilla | Glucose | Prussian blue | GOx | electrochemical | Different current produced by H2O2 | 109 nA/ |
| et al., 2022) | | (PB)/nickel | | | when GOx reacts with glucose | mM |
| | | hexacyanoferrate | | | | |
| (You et | Glucose | PEGDA | GOx | colorimetric | GOx reacts with glucose produces | Color density/ |
| al., 2023) | | | | | gluconic acid generates pH changes | mM |
| | | | | | with color variation | |
| This work | Glucose | CMC-pHEA GelMA- | GOx | Radio frequency | Hydrogel swelling volume variation | 17 Hz/ |
| | | ConA hydrogel | | | when react with glucose | mM |
| | | microneedle | | | | |

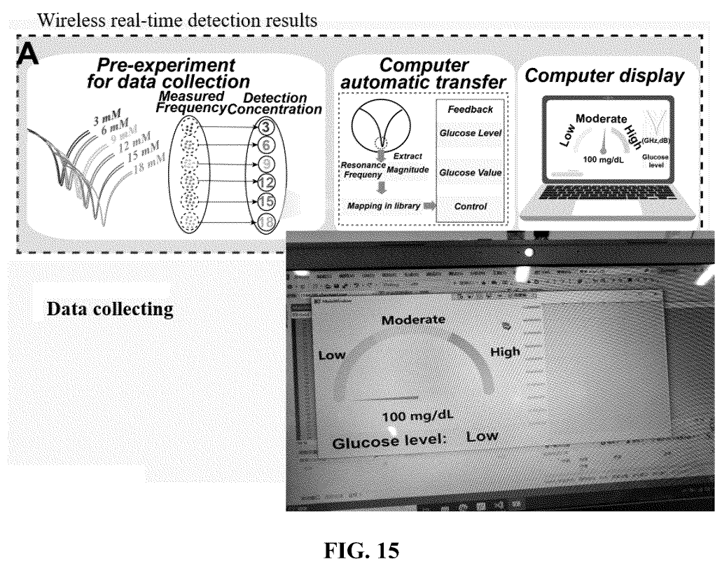

FIG. 15 illustrates a screen showing changes in capacitance of an RF biosensor (LC circuit of LC resonator) with glucose-responsive microneedles→changes in central frequency of the LC measurement resonator→glucose concentration (hypoglycemia/normal/hyperglycemia).

The RF biosensing system using the RF biosensor with the microneedles can measure protein in the skin, blood glucose concentration in capillaries, and fats in vivo. The RF biosensing system using the RF biosensor with the microneedles is connected to a VNA, and the VNA measures glucose concentration in accordance with changes in the resonant frequencies (hypoglycemic frequency f1, normal frequency f2, hyperglycemic frequency f3) of an LC resonator of the RF biosensor and changes in S-parameters (S1, S2, S3), so as to display hypoglycemia, normal, hyperglycemia states, and diagnose diabetes.

### 4. Conclusion

The integrated platform for glucose detection that effectively combines extraction and detection processes has been developed by utilizing an advanced wireless sensing system in conjunction with a swelling hydrogel microneedles. The freeze-dried CMC-pHEA hydrogel network becomes glucose-specific after cross-linking with glucose oxidase and compounds like GelMA-ConA. When the compounds like GelMA-ConA paired with the flexible antenna, this sensing component can tap into the interstitial fluid, and bind to its glucose molecules. As it swells, the degree of swelling corresponds to glucose concentration, leading to noticeable changes in the signal of the wireless reading coil. Employing swelling-based hydrogel microneedles, it ensures swift and precise glucose identification using RF signals, facilitating a minimally invasive and pain-free glucose monitoring.

The microneedles made from glucose-responsive hydrogels can extract interstitial fluid with minimal discomfort.

Glucose detection by hydrogel swelling is measured using RF biosensing technology.

An on-site measurement platform in vivo enables on-site sensing and detection.

The wireless detection platform is capable for passive sensing and remote reading without complex circuits.

The glucose-responsive hydrogel-based microneedles, once minimally invasively attached to the skin inside the body, diagnose diabetes by measuring glucose concentration using the VNA in accordance with changes in capacitances of the sensing signals of the sensing circuit (wireless detection) of the RF biosensor, central frequency shift of the LC resonator of the sensing antenna circuit, and changes in S-parameters.

The RF biosensor using the glucose-responsive hydrogel-based microneedles, which are provided on the bottom of the flexible substrate, is minimally the invasively in contact with epidermis/dermis/hypodermis or skin/fat/muscle in vivo, and the VNA diagnoses diabetes by measuring glucose concentration in accordance with changes in capacitances of the sensing signals of the RF biosensor, central frequency shift of the LC resonator of the sensing antenna circuit, and changes in S-parameters.

The RF biosensor having the glucose-responsive hydrogel-based microneedles, when attached to the skin in vivo, detects glucose in the skin or capillaries and the interstitial fluid (ISF) in subcutaneous fat, complex permittivity and capacitance (C) at different glucose concentrations are changed, and alter and move (shift) the resonant frequencies of the LC resonator of the sensing antenna circuit of the RF biosensor, and also change Su, thereby detecting accurate glucose concentration measurements through measurement of the resonant frequencies.

While specific embodiments of the present invention have been described for illustrative purposes, it should be understood that the present invention is not limited to the specific embodiments and operations described for the purpose of illustrating the technical concept of the present invention. The present invention may be variously modified within the scope of the technical concept and scope of the present invention, as long as it does not deviate from the scope of the claims described below.

What is claimed is:

1. An RF biosensing system using an RF biosensor with glucose-responsive hydrogel-based microneedles, comprising:
a substrate;
a plurality of glucose-responsive hydrogel-based microneedles, which are placed on the bottom of a substrate, and are in minimally invasively contact within the skin in vivo;
an RF biosensor, which includes a sensing antenna circuit and a sensing antenna connected to the glucose-responsive hydrogel-based microneedles provided on the bottom of the substrate and including an LC resonator of an RF biosensor provided on the top of the substrate, and a reader antenna and a reader antenna sensing circuit EM coupled within an effective range and connected through a circular readout coil receiving sensing signals by using RF biosensing and wireless transmission; and
a vector network analyzer (VNA), which measures changes in capacitances of the sensing signals, central frequency shift of the LC resonator, and changes in S-parameters.

2. The RF biosensing system according to claim 1, wherein the microneedles include a plurality of microneedles formed in an n×n matrix structure (where n is a natural number, and n≥1).

3. The RF biosensing system according to claim 1, wherein the microneedles are glucose-responsive hydrogel-based microneedles.

4. The RF biosensing system according to claim 3, wherein the glucose-responsive hydrogel uses CMC-pHEA GelMA-ConA hydrogel.

5. The RF biosensing system according to claim 1, wherein the substrate is one substrate of an FR4: substrate, a flexible substrate, and a ceramic substrate for a broadband antenna of the RF biosensor.

6. The RF biosensing system according to claim 1, wherein the sensing antenna and the reader antenna of the RF biosensor provided on the top of the substrate use RF frequencies in the range of 400 to 3,000 MHz.

7. The RF biosensing system according to claim 3, wherein antibodies attached to the glucose-responsive hydrogel-based microneedles provided on the bottom of the substrate is glucose oxidase, and since glucose reacts with glucose oxidase to produce acid, synthesized CMC-pHEA is used, which is highly sensitive to pH and selectively reacts only with glucose in the swelling state of the hydrogel.

8. The RF biosensing system according to claim 7, wherein the glucose-responsive hydrogel-based microneedles are manufactured using a composite material consisting of CMC, KPS, 2-HEA, PEGDA, gelMA, glucose oxidase, and a photoinitiator to create swelling hydrogel microneedles.

9. The RF biosensing system according to claim 3, wherein the material for the glucose-responsive hydrogel-based microneedles uses biocompatible GelMA (gelatin methacryloyl) hydrogel.

10. The RF biosensing system according to claim 1, wherein the sensing antenna of the RF biosensor is a patch antenna, which includes a central radiator and metal lines of a square-shaped spiral structure in an N-turn structure, and the metal lines are made of gold (Au), silver (Ag), or copper (Cu), and are spaced apart from each other at regular intervals with a line width (W) of 0.1 to 1 mm and line spacing(S) of 0.1 to 1 mm.

11. The RF biosensing system according to claim 10, wherein the microneedles use a 15×15 array, and the penetration depth of the microneedles into the skin is 10 to 100 μm, and
wherein the size of the sensing antenna is 12 mm×12 mm, and the optimum distance for wireless biosensing between the sensing antenna of the RF biosensor and the Readout coil is 1 to 5 mm.

12. The RF biosensing system according to claim 1, wherein when being attached to the skin in vivo, the RF biosensor minimally invasively detects glucose in the skin or capillaries and the interstitial fluid (ISF) in subcutaneous fat, complex permittivity and capacitance (C) at different glucose concentrations are changed, and the changes move the resonant frequencies of the LC resonator of the sensing antenna circuit of the RF biosensor, and also change S₁₁, thereby allowing glucose concentration measurements through measurement of the resonant frequencies.
